(19) United States
(12) Patent Application Publication
(10) Pub. No.: US 2012/0283574 A1
(43) Pub. Date: Nov. 8, 2012

(54) Diagnosis Support System Providing Guidance to a User by Automated Retrieval of Similar Cancer Images with User Feedback

(76) Inventors: Sun Young Park, San Diego, CA (US); Dustin Michael Sargent, San Diego, CA (US); Rolf Holger Wolters, Honolulu, HI (US); Ulf Peter Gustafsson, San Diego, CA (US)

(21) Appl. No.: 13/464,914

(22) Filed: May 4, 2012

Related U.S. Application Data
(60) Provisional application No. 61/518,510, filed on May 6, 2011.

(51) Int. Cl. A61B 1/303; A61B 6/00

(52) U.S. Cl. 600/476

(57) ABSTRACT

The present invention is a diagnosis support system providing automated guidance to a user by automated retrieval of similar disease images and user feedback. High resolution standardized labeled and unlabeled, annotated and non-annotated images of diseased tissue in a database are clustered, preferably with expert feedback. An image retrieval application automatically computes image signatures for a query image and a representative image from each cluster, by segmenting the images into regions and extracting image features in the regions to produce feature vectors, and then comparing the feature vectors using a similarity measure. Preferably the features of the image signatures are extended beyond shape, color and texture of regions, by features specific to the disease. Optionally, the most discriminative features are used in creating the image signatures. A list of the most similar images is returned in response to a query. Keyword query is also supported.

This application claims the priority of U.S. provisional patent application No. 61/518,510, filed on May 6, 2011.

### TECHNICAL FIELD

The present invention generally relates to medical imaging, and more specifically to an image retrieval and user feedback system for the screening, detection, and diagnosis of cervical pre-cancers and cancer.

### BACKGROUND ART

Although this invention is being disclosed in connection with cervical cancer, it is applicable to many other areas of medicine in which image or video data are utilized in the screening, detection, and diagnosis process.

Cervical cancer is the second most common cancer among women worldwide, with about 530,000 new cases and 275,000 deaths per year, accounting for about 9% of all cancers diagnosed in women and 8% of all female cancer deaths, respectively (International Agency for Research in Cancer (IARC), Globocan 2008 database, 2008; incorporated herein by reference). These statistics are troubling since the invasive disease is preceded by premalignant cervical intraepithelial neoplasia (CIN) and, if detected early and treated adequately, cervical cancer is preventable and curable (Ferris, D. G., Cox, J. T., O'Connor, D. M., Wright, V. C., and Foerster, J., “Modern Colposcopy: Textbook and Atlas,” American Society for Colposcopy and Cervical Pathology, 2004; incorporated herein by reference). While the incidence of invasive cervical cancer in the developed world is declining, the incidence of cancer precursors has risen and the disease remains a serious threat to women's health. Worldwide, cervical cancer remains a compelling public health issue, with almost 90% of cervical cases and deaths occurring in developing countries.

Cervical Cancer Screening—The standard cervical cancer screening method is the Papanicolaou (Pap) test, followed by a colposcopy examination if the result of the Pap test is abnormal. The Pap test is a microscopic examination of cells collected from the surface of the cervix. During the test, the size and shape of the nucleus and cytoplasm of the cervical cells discern the abnormalities of cells as a precursor to cervical cancer. The cervical abnormalities that are seen on a Pap test are usually referred to as squamous intraepithelial lesions (SIL) and graded according to low-grade (LSIL), high-grade (HSIL) and possibly cancerous (malignant). However, the grading system (see below) of colposcopy is often also used.

Colposcopy is a systematic visual examination of the lower genital tract (cervix, vulva, and vagina) to identify and rank for biopsy the highest-grade abnormalities. A histopathology analysis of the biopsy samples determines the diagnosis of the cervical abnormalities. The abnormalities that are seen on a biopsy of the cervix are referred to as cervical intraepithelial neoplasia (CIN) and are typically grouped into five categories of CIN 1 (mild dysplasia), CIN 2 (moderate dysplasia), CIN 3 (severe dysplasia), CIS (carcinoma in situ), and invasive carcinoma (cancer). Sometimes in-between categories, such as CIN 1-2 and CIN 2-3, are also used when the abnormalities cannot be exclusively categorized.

Shortcomings of the Pap Test—Although widespread Pap test screening have been effective in reducing the incidence and mortality of cervical cancers in developed countries, it is unclear whether this success can be replicated in developing countries with large female populations, as these countries often lack the sophisticated laboratory equipment, highly trained personnel and financial resources necessary to implement cervical cancer screening programs (Sankaranarayanan, R., Budukh, A. M., and Rajkumar, R.,“Effective screening programmes for cervical cancer in low-and middle-income developing countries,” Bulletin of the World Health Organization 79, pp. 954-962, 2001; Cronje, H. S., “Screening for cervical cancer in developing countries,” International Journal of Gynecology and Obstetrics 84(2), pp. 101-108, 2004; Batson, A., Meheus, F., and Brooke, S., “Chapter 26: Innovative financing mechanisms to accelerate the introduction of HPV vaccines in developing countries,” Vaccine 24, pp. 219-225, 2006; and Gakidou, E., Nordhagen, S., and Obermeyer, Z., “Coverage of cervical cancer screening in 57 countries: low average levels and large inequalities,” PLos Medicine 5(6), pp. 0863-0868, 2008; all incorporated herein by reference).

Furthermore, the accuracy of Pap test screening is limited by a high false negative rate. An extensive meta-analysis of the literature estimated the sensitivity of the standard Pap test screening for CIN 1 and higher to as low as 37% to 84% (Agency for Health Care Policy and Research (AHCPR), “Evaluation of Cervical Cytology,” Evidence Report/Technology Assessment No. 5, Rockville, Md., 1999; incorporated herein by reference). Other studies have estimated the sensitivity of Pap test screening for CIN 2+ and CIN 3+ to be 29-65% and 54-92%, respectively (Gravitt, P. A., Paul, P., Katki, H. A., Vendantham, H., Ramakrishna, G., Sudula, M., Kalpana, B., Ronnett, B. M. and Shah, K. V., “Effectiveness of VIA, PAP, and HPV DNA testing in a cervical cancer screening program in a peri-urban community in Andhra Pradesh, India,” PLoS One 5(10), October 2010; incorporated here by reference).

The low sensitivity of the Pap test is related to poor cell preparation and the limitations of detecting human papillomavirus (HPV), which is a major cause of cervical cancer. The limitations of HPV detection can be addressed by newer screening techniques including HPV DNA (deoxyribonucleic acid) test (Cox, T. and Cuzick, J., “HPV DNA testing in cervical cancer screening: from evidence to policies,” Gynecol. Oncol. 103, pp. 8-11, 2006; incorporated herein by reference) and VIA (visual screening with acetic acid) (Sankaranarayanan. R. and Wesley, R. S., “A practical manual on visual screening for cervical neoplasia,” IARC Technical Publication No. 41, 2003; incorporated herein by reference). The HPV DNA test identifies high risk HPV types and VIA visually detects persistent HPV infections that cause genital warts and cervical cancer. Of these newer screening methods, HPV DNA testing is often unaffordable in low resource setting countries and VIA requires training to accurately determine the severity and extent of cervical abnormalities.

Shortcomings of Colposcopy—Following an abnormal Pap test, colposcopy serves as the critical diagnostic method for evaluating women with potential lower genital tract neoplasias in the developed world. Colposcopy is a challenging clinical procedure largely based on the experience and skill of the colposcopist. As stated by experts from the United States National Cancer Institute (NCI), “optimizing the accuracy of colposcopy and biopsy specimens is one of the leading concerns in the entire cervical cancer screening process”(Jeronimo, J. and Schiffman, M., “Colposcopy at a crossroad,” Obstet. Gynecol. 195, pp. 349-353, 2006, incorporated herein by reference).

The concern regarding colposcopy is based on results from studies that demonstrated the suboptimal accuracy of colposcopy. In the NCI ASCUS (atypical squamous cells of undetermined significance)/LSIL (low grade squamous intraepithelial lesion) Triage Study (ALTS), a sensitivity and specificity of 37% and 90%, respectively, were determined for detecting CIN 3 (Ferris, D. G. and Litaker, M. S., “Prediction of cervical histologic results using an abbreviated Reid Colposcopic Index during ALTS,” Am. J. Obstet. Gynecol. 194(3), pp. 704-710, 2006, and Ferris, D. G. and Litaker, M. S., “Colposcopy quality control by remote review of digitized colposcopic images,” Am. J. Obstet. Gynecol. 191(6), pp. 1934-1941. 2004; both incorporated herein by reference).

Similar poor sensitivity (30%) for diagnosing CIN 2+ was found in a collaborative study from the American Society for Colposcopy and Cervical Pathology (ASCCP) and the NCI (Massad, L. S., Jeronimo, J., Katki, H. A., and Schiffman, M., “The accuracy of colposcopic grading for detection of high-grade cervical intraepithelial neoplasia,” J. Low. Genit. Tract Dis. 13, pp. 137-144, 2009; incorporated herein by reference). Another study reported a sensitivity of 61% and specificity of 94% for discriminating CIN 1 from CIN 2/CIN 3 (Hammes, L. S., Naud, P., Passos, E. P., Matos, J., Brouwers, K., Rivoire, W., and Syrjanen, K., “Value of the International Federation for Cervical Pathology and Colposcopy (IFCPC) terminology in predicting cervical disease,” J. Low. Genit. Tract Dis. 11, pp. 158-165, 2007; incorporated herein by reference).

The accuracy of colposcopy also varies by setting (Cantor, S. B., Cardenas-Turanzas, M., Cox, D., Atkinson, E. N., Nogueras-Gonzalez, G. M., Beck, N. E., Follen, M., and Benedet, J. L., “Accuracy of colposcopy in the diagnostic setting compared with the screening setting,” Obstet. Gynecol. 111(1), pp. 7-14; 2008, incorporated herein by reference) and in women who have previously been treated for cervical neoplasia (Moss, E. L., Dhar, K. K., Byrom, J., Jones, P. W., and Redman, C. W., “The diagnostic accuracy of colposcopy in previously treated cervical intraepithelial neoplasia,” J. Low. Genit. Tract. Dis. 13(1), pp. 5-9, 2009; incorporated herein by reference).

Other studies have demonstrated that 37% to 40% of cervical biopsies taken of colposcopically normal appearing epithelium (not normally biopsied) are diagnosed histologically as CIN 2 or worse (Wentzensen, N., Zuna, R. E., Sherman, M. E., Gold, M. A., Schiffman, M., Dunn, S. T., Jeronimo J, Zhang, R., Walker, J., and Wang, S. S., “Accuracy of cervical specimens obtained for biomarker studies in women with CIN3,” Gynecol. Oncol. 115, pp. 493-496, 2009, and Pretorius, R. G., Zhang, W. H., Belinson, J. L., Huang, M. N., Wu, L. Y., Zhang, X., and Qiao, Y. L., “Colposcopically directed biopsy, random cervical biopsy, and endocervical curettage in the diagnosis of cervical intraepithelial neoplasia II or worse,” Am. J. Obstet. Gynecol. 191(2), pp. 430-434. 2004; both incorporated herein by reference).

In response to suboptimal biopsy site placement, a study from the ALTS trial found that collecting multiple cervical biopsies improves the sensitivity of colposcopy (Gage, J. C., Hanson, V. W., Abbey, K., Dippery, S., Gardner, S., and Kubota, J., “ASCUS LSIL Triage Study (ALTS) Group, Number of cervical biopsies and sensitivity of colposcopy,” Obstet. Gynecol. 108, pp. 264-272, 2006, incorporated herein by reference). Yet, acquiring multiple biopsies from seemingly healthy tissue causes an increased risk of infection, bleeding, patient discomfort, anxiety, procedural time and cost.

The ALTS trial has further demonstrated the inherent value of cervical imagery databases for cervical cancer screening, detection, and diagnosis. However, existing colposcopic imagery databases relying mostly on digitized film-based photographs suffer from low-quality, low-definition imagery lacking adequate standardization. For example, colposcopists and reviewers in one study using digitized cervical images under-diagnosed 16% and 25% of subjects, and over-diagnosed 45% and 20% of subjects compared with histopathology, respectively (Ferris, D. G. and Litaker, M. S., “Colposcopy quality control by remote review of digitized colposcopic images,” Am. J. Obstet. Gynecol. 191(6), pp. 1934-1941. 2004; incorporated herein by reference).

From a device standpoint, colposcopes being used today have not kept pace with the advances in information technology. Most existing colposcopes are analog and do not provide diagnostic enhancement features to aid in colposcopic exams. Nor are they capable of seamless connectivity with electronic health records based on standardized systems and protocols such as the Picture Archiving and Communication System (PACS), Digital Imaging and Communications in Medicine (DICOM), and Veterans Health Information Systems and Technology Architecture (VistA).

Consequently, innovative solutions to provide accurate, standardized, screening, detection, and diagnosis support systems for cervical pre-cancer and cancer detection coincide with the need to improve the efficacy and cost-effective implementation of screening techniques such as the Pap test, HPV DNA tests, and VIA, as well as the overall diagnostic accuracy of the currently subjective procedure of colposcopy. Furthermore, as the current standard of care relies on several consecutive tests and exams separated in time from days to weeks, and involves a number of experts for each test and exam, solutions to decrease the total test and exam time and overall cost are of high importance, both in the developed and the developing worlds.

### DISCLOSURE OF THE INVENTION

The present invention of a cervical cancer image retrieval and user feedback system, described herein and more fully below, is a global information sharing and clinical reference diagnosis support system providing cost-effective, time-effective and objective screening, detection, and diagnosis support for cervical pre-cancer and cancer.

The diagnosis support system of the present invention provides global access to standardized high-resolution cervical images, colposcopy impressions and annotations from expert colposcopists, histopathology diagnosis and annotations from pathologists, patient biographical information, treatment history, hospital and physician information, screening, detection, and diagnosis results, as well as advanced analysis and feedback tools in a convenient database, providing the means to increase the proficiency and diagnostic power of all practitioners independent of their expertise and location. The diagnosis support system enables expert level cervical cancer screening, detection, and diagnosis to be efficiently and accurately delivered in every location and to every practitioner.

The diagnosis support system is an automated solution to improved patient treatment and improved diagnostic outcome by empowering practitioners to make knowledge-based decisions using the collective experience of all practitioners from all exams. The system provides decision support from expert colposcopists and pathologists to every practitioner performing cervical cancer screening, detection, and diagnosis. Practitioners can query the diagnosis support system for cases similar to their patient and obtain annotated images, diagnostic outcomes, and case reports from similar patients treated by expert colposcopists. This allows less trained or experienced practitioners to provide better healthcare under expert guidance. It also provides the means to reduce per patient cost by increasing comparative effectiveness of medical treatment and practices.

The diagnosis support system of the present invention replaces traditional health records and examination reports by providing universal access to standardized electronic health records for cervical cancer patients. The system provides at least DICOM/PACS/VistA compliance, ensuring uniformity and portability between devices from different manufacturers and in different countries. Upon completion of a cervical cancer exam, the digital images, colposcopy impressions and histopathology reports are automatically uploaded to the diagnosis support system and added to the patient's electronic health record. This provides a complete health and examination history that is instantly accessible to all physicians and clinics connected to the diagnosis support system, further improving the accuracy of diagnosis and reliability of treatment decisions. With all this expert knowledge instantly accessible to every practitioner, the diagnosis support system also provides the means to possibly collapse the time-consuming and costly procedures of screening, colposcopy exam, and histopathology analysis into one single exam in which the patient is screened, diagnosed, and treated at the same visit.

The contents of the diagnosis support system are standardized and defined by world experts in colposcopy and cervical cancer as well as the individual practitioners and are available on-line via telemedicine or stored locally as a subset. The diagnosis support system provides for effective telemedicine and the foundation for highest quality education, training and continuing education. Procedural guidelines and training aid the practitioner in the colposcopic exam, expediting the procedure and reducing costs. This training and education are made possible by so called information centers for digital colposcopy in which expert colposcopists and pathologists are available for evaluation of images and data, and diagnostic decision support.

The main objective of the diagnosis support system is to enhance a practitioner's effectiveness during the cervical cancer screening, detection, and diagnosis in both procedure and outcome. This is the first time that a database for colposcopy will have clinical utility based on the ability of the clinician to access cumulative knowledge through automated guidance rather than a resource solely as a science research document. The automation of the diagnosis support system and the knowledge base it contains are developed and enhanced via work done at the information centers and medical research organizations and provided to the practitioners with transparency for real time assistance and guidance in the practical issues encountered including but not limited to:
Case-to-case comparisons using side-by-side imagery deemed similar by the diagnosis support system;
Being able to have access within seconds to usable information and knowledge specific to the case at hand;
Improve skill as a colposcopist through access to the collective knowledge of the whole field;
Normalization of annotations by algorithms leading to increased standardization of the exams;
Expansion of the available data by the use of advanced image processing;
Automatic advisory and statistical comparison relative to the knowledge base to improve the ability of the physician to advise patient of outcome of exam;
Ability to track patient health over time for abrupt changes or progression of conditions; and
Access to the best and most relevant research and recommendations from the medical field.

The diagnosis support system centralizes global knowledge to solve health problems. By automating costly and time consuming tasks such as image annotation, storage and retrieval, the diagnosis support system helps physicians improve their health care standards and facilitates access to data for research into future medical breakthroughs. Applying machine learning and data mining techniques to the vast amount of accumulated data, the diagnosis support system can discover patterns that will lead to improved health care planning and quality control of examinations and diagnoses.

### BRIEF DESCRIPTION OF THE DRAWINGS

FIG. 1 is a conceptual diagram of the global information sharing and clinical reference diagnostic support system of the present invention.

FIG. 2 is a conceptual diagram of the image retrieval functionality.

### BEST MODE FOR CARRYING OUT THE INVENTION

The present invention of a cervical cancer image retrieval and user feedback system, described herein and more fully below, is a global information sharing and clinical reference diagnosis support system providing cost-effective, time-effective and objective screening, detection, and diagnosis support for cervical pre-cancer and cancer.

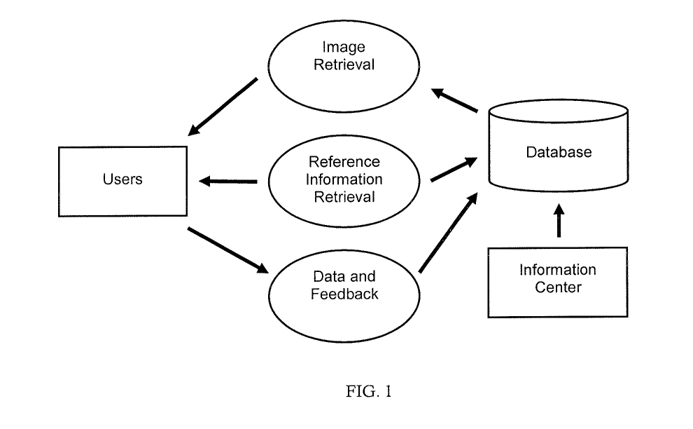

A conceptual diagram of the diagnosis support system of the present invention is shown in FIG. 1. The diagnosis support system is an automated database of cervical digital imagery with associated meta-data in terms of patient biographical information, treatment history, hospital and physician information, screening, detection, and diagnosis results, colposcopy impression and annotations, histopathology diagnosis and annotations, and advanced analysis and feedback tools. The diagnosis support system incorporates the important functionalities of reference information and image retrieval. The diagnosis support system also stores and transmits the imagery and data from and to the user. Furthermore, the diagnosis support system incorporates user feedback to increase the functionality of the database and improve the performance of the information and image retrievals. The diagnosis support system also integrates information centers in which expert colposcopists and pathologists perform evaluations of images and data, and provide diagnostic decision support to the user in real-time.

The diagnosis support system can be deployed as a standalone database application or as a global information sharing system. The standalone database application includes the full functionality of the diagnosis support system, but applied to images and data stored on a local computer or a local network only. For the global information sharing system, images and data are automatically uploaded to a repository connected to the diagnosis support system, and added to the patient's electronic health record. Image and data retrieval can be performed on every image in the repository.

The diagnosis support system provides a complete health record and examination history that is instantly accessible to all physicians and clinics connected to the system's network. This empowers every practitioner to make knowledge-based decisions using the collective experience of all practitioners from all exams (in the system), improving the accuracy of diagnosis and reliability of treatment decision of every exam. With all this expert knowledge instantly available to the practitioner, the diagnosis support system provides the means to collapse the time-consuming and costly procedures of screening, colposcopy exam, and histopathology analysis into one single exam in which the patient is screened diagnosed, and treated at the same time. Furthermore, with complete health and records and examination history, the diagnosis support system provides the ability to track patient health over time for changes or progression of conditions. And with every user (practitioners to experts) contributing to the diagnosis support system's knowledge, the system provides access to the best and most relevant research and recommendations from the medical fields.

With a large set of image and data information, the diagnosis support system benefits from the use of cloud computing (Mell, P. and Grance, T., “The NIST definition of cloud computing,” National Institute of Standards and Technology, Special Publication 800-145, 2011; incorporated herein by reference). The cloud provides the storage and database functionality required for the diagnosis support system at a low cost and eliminates the overhead associated with establishing a distributed network. Users can connect to the diagnosis support system through a browser-based application, which preferably includes all, or parts based on the user's preferences, of the system's functionality. A web-based service using cloud computing provides the scalability and availability required for a global information sharing and clinical reference diagnostic support system of the present invention.

Image Data—The cervical image data stored in and retrieved from the database are preferably acquired by a high-resolution digital colposcope (such as described in co-pending, commonly assigned patent application entitled “High resolution digital video colposcope with built in polarized LED illumination and computerized clinical data management system,” U.S. patent application Ser. No. 12/291,890 and International Patent Application #PCT/US2008/012792, both filed Nov. 14, 2008 and both incorporated herein by reference) to ensure that diagnostically relevant features are accurately captured in the digital imagery.

The image data is preferably also standardized in terms of color (such as described in commonly assigned patent entitled “Method of automated image color calibration,” U.S. Pat. No. 8,027,533, filed Mar. 19, 2008) and quality (such as described in co-pending, commonly assigned patent applications entitled “Method of image quality assessment to produce standardized imaging data,” U.S. patent application Ser. No. 12/075,910, filed Mar. 14, 2008; and “A method to provide automated quality feedback to imaging devices to achieve standardized imaging data,” U.S. patent application Ser. No. 12/075,890, filed Mar. 14, 2008; both incorporated herein by reference) to ensure that the images are independent of the digital acquisition used and the localization of where the images are acquired.

As the use of acetic acid is a fundamental part in the visual discrimination of normal and pre-cancerous tissue, the cervical image data preferably also includes images acquired before and after the application of acetic acid. Potential pre-cancerous epithelial cells in the cervix typically turn white after the application of acetic acid. Virtually all cervical cancer lesions become a transient and opaque white color following the application of 5% acetic acid. This whitening process occurs visually over several minutes and subjectively discriminates between pre-cancerous and normal tissue.

In order to improve the functionality and provide support for user-specific needs, the database preferably also incorporates user-defined imagery. The images and data are preferably also handled, stored, printed, and transmitted according to the DICOM (Digital Imaging and Communications in Medicine) standard, and the database preferably employs the picture archiving and communication system (PACS). Furthermore, the database design is preferably compliant with large scale information systems built around an electronic health record, such as the Veterans Health Information Systems and Technology Architecture (VistA). This ensures quick and efficient storage and retrieval of images and portability between different imaging modalities and health care providers, and devices from different manufacturers.

Annotations—The colposcopy impressions and annotations as well as the histopathology diagnosis and annotations are preferably provided according to standard colposcopy and pathology procedures (Ferris, D. G., Cox, J. T., O'Connor, D. M., Wright, V. C., and Foerster, J., “Modern Colposcopy: Textbook and Atlas”, American Society for Colposcopy and Cervical Pathology, 2004; and Burghardt, E., Pickel, H. and Girardi, F., “Colposcopy—Cervical Pathology, Textbook and Atlas”, Thieme, 1998; both incorporated herein by reference).

The colposcopy annotations could also include a detailed set of annotations with any or all of the following cervical tissue types and diagnostic features: adequacy of exam, biopsy sites, cervix, cervical os, squamo-columnar junction, squamous epithelium, columnar epithelium, metaplasia, acetowhite translucent opacity, acetowhite intermediate opacity, acetowhite opaque opacity, acetowhite flat white gloss, acetowhite shiny white gloss, acetowhite peri-glandular cuffings, acetowhite gray color, acetowhite yellow color, acetowhite black color, fine mosaic, coarse mosaic, fine punctation, coarse punctation, parallel vessels, network vessels, regular lesion margin shape, irregular lesion margin shape, diffuse lesion margin demarcation, distinct lesion margin demarcation, internal lesion margin, peeling lesion margin, satellite lesion margin, raised contour, irregular contour, glands, asperities, nabothian follicles, ulceration, petechia, severe inflammation, condyloma, deciduosis, polyps, parakeratosis, hyperkeratosis, mucus, blood, LSIL, HSIL, CIN 1, CIN 1-2, CIN 2, CIN 2-3, CIN 3, CIS, and invasive cancer.

The histopathology annotations could also include a detailed set of annotations with any or all of the following features (such as described in the co-pending, commonly assigned patent application entitled “Process for preserving 3D orientation to allowing registering histopathological diagnoses of tissue to images of that tissue,” U.S. patent application Ser. No. 12/587,614, filed Oct. 8, 2009; incorporated herein by reference): normal squamous, normal glands, immature squamous metaplasia, reactive glandular epithelium, inflammation, atypical immature metaplasia, over gland extension, LSIL, HSIL, CIN 1, CIN 1-2, CIN 2, CIN 2-3, CIN 3, squamous carcinoma, adeno-carcinoma in situ, surface epithelium, basement membrane, destroyed surface epithelium, and no epithelium.

In order to improve the functionality and provide support for user-specific needs, the database preferably also incorporates user-defined annotations.

Other Information—Patient biographical information, treatment history, hospital and physician information are preferably also provided according to standard medical procedures.

Biographical and treatment history would preferably include all or part of the following: name (or unique patent number), age, race, reason for screening, reason for colposcopy, reason for biopsy, cytological results, history of CIN 1, history of CIN 2, history of CIN 3, gravidity, parity, history of vaginal delivery, use of birth control, menstrual status (pre-menopausal, menopause, post-menopausal, other), history of sexually transmitted disease (HPV, gonorrhea, syphilis, chlamydia, HIV/AIDS, other), prior cervical treatment and procedures, smoking history, current and prior drug use, family history of cancer, complications, and management recommendations. It should be noted that to ensure patient confidentiality, any personal information regarding the patient is only available to the assigned physician of the patient. No other users would have access to any personal information.

Hospital information could include the name, address, screening and/or treatment of cervical pre-cancer and cancer, number of clinics, number of colposcopists, and number of pathologists.

Physician information could include name, medical field, disease specialization, expert or general practitioner, and years of experience.

In order to improve the functionality and provide support for user-specific needs, the database could also incorporate user-defined information.

Reference Information Retrieval—Reference information retrieval is the process in which the user queries the database based on text input relating to all or part of the meta-data.

The output of the search could display all information for every patient fulfilling the search criteria. The text input could, for example, be one text entry such as find and display the information for all patients that have CIN 1. A more meaningful search would be to combine different text inputs, such as find and display the information for all patients that smoke, have a family history of cancer, and have CIN 2 or higher.

The output of the search could also display a subset of the information retrieved. For example, find all patients with colposcopy annotations and who have CIN 3, but only display the images and the annotations for the patients.

In order to improve the functionality and provide support for user-specific needs, user-specified search metrics could also be incorporated.

Information Centers—With the use of digital colposcope systems, nurses, or technicians can use these devices to acquire digital imagery of a large number of patients. The images are then integrated into the database, and can also be sent to the information center, where they are reviewed by experts in colposcopy and pathology. The physical location of the experts is not important as long as they can communicate with the digital colposcope system and the practitioner. The experts then return a diagnosis to the digital colposcope system, or takes control of the system remotely for direct examination. This allows the existing experts to efficiently perform a large number of simultaneous diagnoses, independent of the physical location of the patients. Instead of requiring multiple visits, diagnosis is performed immediately, without the associated cost with current screening programs.

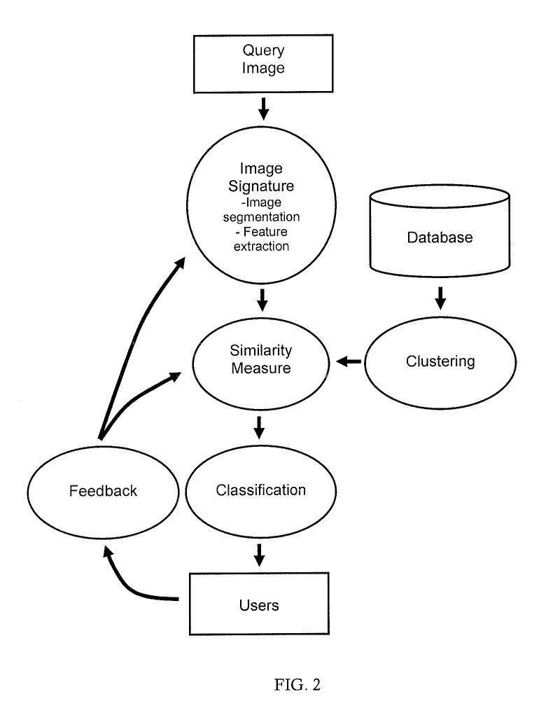

Image Retrieval—The image retrieval, as conceptually described in FIG. 2, provides two basic functionalities: 1) meta-data based image retrieval using patient biographical information, treatment history, hospital and physician information, annotations, and diagnostic results; and 2) content-based image retrieval using automatically generated features. The general idea of image retrieval is that a user queries the database by providing a query image, and the system returns images from the database that are similar in appearance to the query image. This function is in way similar to the information centers, except that the feedback or diagnosis is provided automatically using a computer system, without the need for an expert being available remotely.

For meta-data based image retrieval, diagnostic features are extracted from the database images based on the meta-data information contained in the database for each image. Although all of the meta-data contained in the database could be used, the following diagnostic features are preferably always used: colposcopic impression (normal, CIN 1, CIN 2, CIN 3, CIS, and cancer), histopathology diagnosis (normal, CIN 1, CIN 2, CIN 3, CIS, and cancer), acetowhite lesion size, acetowhite intensity, punctation (coarse and fine), mosacism (coarse and fine), atypical vessels, and lesion margins. Clustering is then applied to the database images to group the extracted diagnostic features. An overlapping clustering algorithm is applied to enable assigning each patient image to multiple clusters so as not to constrain the images to one cluster only. A similarity measure is then applied and returns a ranked list of similar images to the user. The user can then optionally provide feedback concerning the relevance of the search result.

For content-based image retrieval, image signatures (described below) based on color, texture, shape, and other features contained in the image are first automatically computed to describe the query image. Then a similarity measure is used to compare the query image with images from the database. The database images were preferably previously clustered and classified based on image signature and other visual content, so the query image need not be compared with every image in the database. As for the meta-data based search, an overlapping clustering algorithm is also preferably applied to enable assignment of each patient image to multiple clusters, so as not to constrain the images to one cluster only. The similarity measure returns a ranked list of similar images to the user, who optionally provides feedback concerning the relevance of the search results to the query. The user feedback is used to improve the image signature and similarity measure.

For the image retrieval, two main types of image searches can be identified: query-by-keyword, and query-by-example-image. Query-by-keyword is the more difficult of these two problems, as it requires image understanding to translate words into visual concepts and must deal with the many different ways in which a given image can be interpreted. Therefore, systems handling this type of search are often trained only to recognize images of a small number of object categories. In the present invention, previously obtained expert user relevance feedback has preferably been incorporated to provide and improve the functionality of query-by-keyword. By contrast, query-by-example-image can be cast as an entirely computational problem. Given a quantitative image description based on image features, such as image signature, a query can be answered by generating the description of the query image and then searching the database for its nearest neighbors in feature space. With cervical imagery as the query image, quantitative descriptions using both general and specific image analysis algorithms can be applied.

The following sections describe in more detail the preferred embodiments of the image retrieval functionality in terms of image signatures, similarity measure, clustering, classification, user relevance feedback, and user-specified search metrics. The design is general-to-specific in which general medical and computer vision algorithms provide the framework of the invention. This framework is then augmented with disease-specific image processing algorithms to provide specialized cervical image analysis functionality. These specialized analysis tools provide a basis for also developing similar tools for other types of medical images. Thus, the design of the present invention is ideally suited for all medical modalities in which images or videos are viewed or acquired, and used in the screening, detection, and diagnosis process.

Image Signatures

Describing images mathematically is a key component in an image retrieval system. Image description, or signatures, describe images quantitatively and provides the basis for comparing different images. Image description usually involves two tasks: segmentation of the image into regions, followed by the extraction of features in each segmented region (“local features”), such as shape, color, texture, and other features contained in the region. A large feature set is preferably extracted for each region, and then features are selected to determine a reduced set of the features that best distinguish between regions, in order to maximize performance and eliminate redundancy.

Image Segmentation: Image segmentation is applied to delineate image regions and assist in the extraction of the local region-based features. The preferred embodiment of the present invention utilizes a mean shift image segmentation algorithm as originally described by Comaniciu and Meer (Comaniciu, D. and Meer, P., “Mean shift: a robust approach toward feature space analysis,” IEEE Transactions on Pattern Analysis and Machine Intelligence 24(5), pp. 603-619, 2002; incorporated herein by reference). Mean shift is an adaptive clustering algorithm which does not require the number of clusters to be specified in advance, and which can provide segmentation in real-time. For each data point, mean shift locates the nearest stationary point of a kernel function using an iterative process. Data points which converge to the same stationary point are clustered in the same cluster.

In order to expand the functionality of image segmentation, other image segmentation methods such as k-means (MacQueen, J. B., “Some methods for classification of multivariate observations,” Proceedings of the 5^{th} Berkeley Symposium on Mathematical Statistics and Probability, pp. 281-297, University of California Press, 1967; and Steinhaus, H., “Sur la division des corps materiels en parties,” Bull. Acad. Polon. Sci. 4(12), pp. 801-801, 1957; both incorporated herein by reference), expectation maximization (EM) (Carson, C., Thomas, M., Belongie, S., Hellerstein, J. M., and Malik, J., “Blobworld: A system for region-based image indexing and retrieval, Lecture Notes in Computer Science,” pp. 509-516, 1999; and Carson, C., Belongie, S., Greenspan, H., and Malik, J., “Blobworld: image segmentation using expectation-maximization and its application to image querying,” IEEE Transactions on Pattern Analysis and Machine Intelligence 24(8), pp. 1026-1038, 2002; both incorporated herein by reference), and graph-cut clustering (Wu, Z. and Leahy, R., “An optimal graph theoretic approach to data clustering: theory and its application to image segmentation,” IEEE Transactions on Pattern Analysis and Machine Intelligence 15(11), pp. 1101-1113, 1993; Shi, J. and Malik, J., “Normalized cuts and image segmentation,” IEEE Transactions on Pattern Analysis and Machine Intelligence 22(8), pp. 888-905, 2000; and Joachims, T., “Transductive learning via spectral graph partitioning,” International Conference on Machine learning 20, pp. 290-297, 2003; all incorporated herein by reference) can also be preferably employed. A drawback of the k-means and EM segmentation approaches compared to the mean shift segmentation algorithm is that these approaches require the number of clusters to be specified. The mean shift segmentation algorithm also enjoys a performance advantage over graph-cut clustering in that the computational cost required by such algorithms limits their use to images of small size.

For meta-data image retrieval, image segmentation is automatically achieved with the colposcopy and histopathology annotations, meaning that a segmentation algorithm is not required. However, the segmentation contained in these annotations can preferably also be used to provide further segmentation of the cervical images for the content-based image retrieval.

Feature Extraction: Once an image has been portioned into regions, each region is then described using color, texture, shape, and other features that produces a set of vectors to describe each region. The individual features are local, as they are used to describe the regions of the entire image and are computed in a neighborhood surrounding a pixel or sub-pixel position in the image. Global features can also be used but since a single signature computed for an entire image cannot sufficiently capture the important properties of individual regions, they do not provide the discriminating power required for the present invention.

Color: Color features are preferably computed using generic color spaces such as RGB (Red Green Blue) and CMYK (Cyan Magenta Yellow, Black) but also perceptually uniform color spaces such as CIE (International Commission on Illumination) L*a*b* and L*u*v*, and approximately perceptually uniform color spaces such as HSV (Hue Saturation Value) and HSL (Hue Saturation Luminance. This allows a large feature set to be extracted and enhances the utility of using color features in the image signature.

The color features extracted include but are not limited to the mean, standard deviation, and entropy for each color band, and the ratio for pairs of color bands (such as R/B, R/G, G/B, etc.) for both individual images and the differences between images. The perceptually and approximately perceptually uniform color corresponds better to human vision than the standard color spaces. Difference measures are comparable to human perception in these color space, allowing for more meaningful difference computations between colors by treating the coordinates as a three-vector and computing their Euclidean distance. This makes these color spaces particularly useful in comparing images using color as a feature. Using these spaces, color distribution features and spatial color descriptors are also preferably included in the feature selection process. Additionally, by preferably utilizing standardized imagery as described previously, the robustness of using color features in the similarity measure can be enhanced.

Texture: Texture features measure the patterns and granularity of the surfaces in an image.

Texture feature methods preferably employed in the present invention include but are not limited to Harris corner detector (Harris, C. and Stephens, M., “A combined corner and edge detector,” Fourth Alvey Vision Conference, pp. 147-151, 1988; incorporated herein by reference), Scale Invariant Feature Transform (SIFT) (Lowe, D., “Distinctive image features from scale-invariant keypoints,” Int. J. Comput. Vision 60, pp. 91-100, 2004; and Brown, M. and Lowe, D., “Invariant features from interest points groups,” British Machine Vision Conference, pp. 656-665., Cardiff, Wales, 2002; both incorporated herein by reference), gradient location and orientation histogram (Mikolajczyk, K. and Schmid, C., “A performance evaluation of local descriptors,” IEEE Transactions on Pattern Analysis and Machine Intelligence 27(1), pp. 1615-1630, 2005; incorporated herein by reference), Speeded-Up Robust Features (SIFT) (Bay, H., Ess, A., Tuytelaars, T., and Van Gool, L., “SURF: speeded up robust features,” Computer Vision and Image Understanding 110(3), pp. 346-359, 2008; and Terriberry, T., French, L., and Helmsen, J., “GPU accelerating speeded-up robust features,” Proceedings of the 4^{th} International Symposium on 3D Data Processing, Visualization and Transmission (3DPVT'08), pp. 355-362, 2008; both incorporated herein by reference), and affine invariant region descriptors (Mikolajczyk, K. and Schmid, C., “A performance evaluation of local descriptors,” IEEE Transactions on Pattern Analysis and Machine Intelligence 27(1), pp. 1615-1630, 2005; and Matas, J., Chum, O., Urba, M., and Pajdla, T., “Robust wide baseline stereo from maximally stable extremal regions,” British Machine Vision Conference, pp. 384-396, 2002; both incorporated herein by reference).

An important factor to consider in the use of texture features is that they are computed in a neighborhood surrounding a point of interest. The point of interest may be a keypoint detected by an algorithm such as SURF, or the center of a region from image segmentation. A key characteristic of the present invention is to be able to identify images as similar if they view the same scene or objects, even if they view the scene from different positions and angles, or the scale and orientation of objects has changed. Therefore, the surrounding neighborhood must be carefully chosen, and features must be computed such that they are invariant to many types of variations that can occur in medical images.

The preferred embodiment of the present invention is to utilize a medical feature detector and descriptor that is invariant to changes in scale, contrast, and rotations about the viewing direction of the camera (such as described in Sargent, D., Chen, C.-I., Tsai, T., Koppel, D., and Wang, Y.-F., “Feature detector and descriptor for medical images,” Proc. SPIE 7259, pp. 72592Z-1—8, 2009; incorporated herein by reference). The present invention expands on this method by extending the feature detector and descriptor to work with region-based image signatures. This is accomplished by separating the extracted interest points according to the region in which they are contained, as well as by adding statistical analyses of the features in each region. These features includes the density of features in a region, which measures the overall amount of texture in that region, and the variance of the observed features, which provides a measure of the entropy or disorder within a region.

One weakness of the described feature descriptor is that it does not provide invariance against 3D rotations; that is, general rotations that change the orientation of the image plane as opposed to 2D rotations in which the camera only rotates about its viewing axis. While these motions may not occur frequently in some medical applications, they are common in fields relying on video data such as colonoscopy. This issue must be addressed to provide a general framework that can be extended to other areas beyond cervical images. The present invention therefore incorporates affine invariant feature descriptors (Mikolajczyk, K. and Schmid, C., “Scale and affine invariant interest point detectors,” International Journal of Computer Vision 60(1), pp. 63-86, 2004; incorporated herein by reference) into the image signature. An affine transformation is any linear transformation plus a translation. Affine transformations preserve collinearity and ratios of distances along a line. The linear transformation can be any combination of rotation, scaling, and shear. The affine invariant features provide additional degrees of invariance at the cost of increased computational complexity.

Shape: Shape features are constructed by extracting contours and curves from images. Shape feature methods preferably employed in the present invention include but are not limited to local shape descriptors (Petrakis, E. G. M., Diplaros, A., and Milios, E., “Matching and retrieval of distorted and occluded shapes using dynamic programming,” IEEE Transactions on Pattern Analysis and Machine Intelligence 24(11), pp. 1501-1516, 2002; and Latecki, L. J. and Lakamper, R., “Shape similarity measure based on correspondence of visual parts,” IEEE Transactions on Pattern Analysis and Machine Intelligence 22(10), pp. 1185-1190, 2000; both incorporated by reference), shape context (Belongie, S., Malik, J., and Puzicha, J., “Shape matching and object recognition using shape contexts,” IEEE Transactions on Pattern Analysis and Machine Intelligence 24(4), pp. 509-522, 2002; incorporated herein by reference), and Fourier descriptors (Bartolini, I., Ciaccia, P., and Patella, M., “Using the time warping distance for Fourier-based shape retrieval”, University of Bologna, 2002; and Bartolini, I., Ciaccia, P., and Patella, M., “Warp: Accurate retrieval of shapes using phase of Fourier descriptors and time warping distance,” IEEE Transactions on Pattern Analysis and Machine Intelligence 27(1), pp. 142-147, 2005; both incorporated herein by reference).

As for texture, shape matching should also be invariant to transformations such as scaling, translation, and rotation. The literature includes accounts in which a dynamic programming approach referred to as dynamic time warping has been applied to achieving these invariant conditions (Bartolini, I., Ciaccia, P., and Patella, M., “Warp: Accurate retrieval of shapes using phase of fourier descriptors and time warping distance,” IEEE Transactions on Pattern Analysis and Machine Intelligence 27(1), pp. 142-147, 2005, incorporated herein by reference). However, in medical applications, shapes can often deform because images contain human tissue and organs rather than rigid structures. The present invention therefore preferably incorporates invariance under small deformations only into a general dynamic programming approach (Adamek, T. and O'Connor, N. E., “A multiscale representation method for nonrigid shapes with a single closed contour,” IEEE Transactions on Circuits and Systems for Video Technology 14(5), pp. 742-753, 2004; incorporated herein by reference).

Feature Selection: With a large set of features extracted, feature selection is applied to determine the most discriminative features, eliminate redundancy, and improve the speed of similarity measure computation. The present invention preferably employs methods such as principal component analysis and genetic algorithms (Mitchell, M., “An Introduction to Genetic Algorithms,” Bradford Books, 1996; incorporated herein by reference), although other methods providing similar outcome can be used. Genetic algorithms are stochastic global optimization algorithms, often applied to problems that are difficult to solve with traditional optimization using analytical properties of the problem.

Extension of Image Signatures using Image Processes to Extract and Classify Tissue Types and Diagnostic Features: The general image signatures are determined by segmentation and local feature extraction using color, texture, and shape. This general framework is extended into cervical-specific image signatures by integrating cervical image processing and detection algorithms that extract and classify tissue types and diagnostic features of the cervix such as anatomical features (cervix region, cervical os, columnar epithelium, squamous epithelium, and metaplasia), vessels (mosaic, punctation, and atypical), aceotowhite color and opacity, lesion margins, CIN 1, CIN 2, CIN 3, CIS, and invasive carcinoma. The present invention preferably extracts and classifies these diagnostic features according to the methods disclosed in commonly assigned patents and co-pending, commonly assigned patent applications entitled “Uterine cervical cancer computer-aided diagnosis (CAD),” U.S. Pat. No. 7,664,300, filed Aug. 15, 2006; “Computerized image analysis for acetic acid induced cervical intraepithelial neoplasia,” U.S. Pat. No. 8,131,054, filed Aug. 4, 2008; “Method for detection and characterization of atypical vessels in cervical imagery,” U.S. Pat. No. 8,090,177, filed Aug. 1, 2008; “Methods for tissue classification in cervical imagery,” U.S. patent application Ser. No. 12/587,603 and International Patent Application #PCT/US2009/005547, both filed Oct. 9, 2009; “Methods for enhancing vascular patterns in cervical imagery,” U.S. patent application Ser. No. 12/228,739 and International Patent Application #PCT/US2008/009777, both filed Aug. 15, 2008; and “Image analysis for cervical neoplasia detection and diagnosis,” U.S. patent application Ser. No. 13/068,188 and International Patent Application #PCT/US2011/000778, filed May 3, 2010; all incorporated herein by reference). However, other methods that automatically extract and classify cervical tissue and diagnostic features into regions can also be used.

Since these algorithms segment regions of the cervix, they integrate seamlessly into the general image signature framework. The vectors of the tissue types and diagnostic features extracted by the image processing and detection algorithms (“feature vectors”) are added to the general region vectors, and an optimal weighting of the different types of vectors are determined as part of the similarity measures described in a following section.

Extension of Image Signatures using Annotations: In addition to the extension of the general framework by integrating cervical image processing and detection algorithms, the content-based image retrieval can be further expanded by also incorporating the colposcopy and histopathology annotations as described earlier. The annotations would preferably also include parts or all of the tissue types and diagnostic features extracted and classified with the cervical image processing and detection algorithms. In this way, the annotations provide another layer to the image signatures, and also provide the means to verify the performance of the image processing and detection algorithms.

Similarity Measures

Similarity measures are used to compare two images using their signatures, or features, and are another key component of the present invention. Good similarity measures for images preferably agree with human interpretation, and are robust and efficient (Datta, R., Joshi, D., Li, J., and Wang, J. Z., “Image Retrieval: Ideas, Influences, and Trends of the New Age,” ACM New York, 2008; incorporated herein by reference).

The present invention preferably compares two images utilizing a combination of relation- and content-based similarity measures. Relation-based similarity measures assess the similarity between regions in terms of the relation between neighborhood regions. Content-based similarity measures assess the similarity between regions based on their content, or features, preferably with some weighting scheme for the different features.

Relation-based Similarity: For relation-based similarity, the following measures are preferably employed.

Dice's coefficient (Dice, L. R., Measures of the amount of ecologic association between species, Ecology 26(3), pp. 297-302, 1945; incorporated herein by reference) is a similarity measurement over sets. Let Relₓ denote the set of relationships of region or image x; then, the relationship similarity Sim_{Rel}(x,y) of two regions or images x and y is calculated according to:

$${Sim}_{Rel\_dice} ( x , y ) = \frac{2 \vert {Rel}_{x} ⋂ {Rel}_{y} \vert}{\vert {Rel}_{x} \vert + \vert {Rel}_{y} \vert} \qquad ( 1 )$$

Jaccard Index, also known as Jaccard's similarity coefficient (Bank, J. and Cole, B., Calculating the Jaccard similarity coefficient with map reduce for entity pairs in wikipedia, The Web Lab, Cornell University, 1996; incorporated herein by reference), is a statistical measure of similarity for two sets A and B and is defined as the size of the intersection divided by the size of the union of the sets according to:

$$J ( A , B ) = \frac{\vert A ⋂ B \vert}{\vert A ⋃ B \vert} \qquad ( 2 )$$

The Jaccard index can be applied to assessing the similarity between two regions or images. By defining sets A and B as relationships Relₓ and Rel_{y}, the relationship similarity Sim_{Rel}_{_{—}}_{Jaccard}(x,y) of two regions or images x and y can be determined or measured according to:

$${Sim}_{Rel\_Jaccard} ( x , y ) = \frac{\vert {Rel}_{x} ⋂ {Rel}_{y} \vert}{\vert {Rel}_{x} ⋃ {Rel}_{y} \vert} \qquad ( 3 )$$

The Jaccard index can preferably also be used for content-based similarity.

Normalized adjacency matrix (Jetchov, N., Similarity measures for smooth web page classification, Master's Thesis, Darmstadt University, 2007; incorporated herein by reference) is defined as:

S=D^{-1/2}WD^{-1/2} (4)

where W is the n×n adjacency matrix with n being the number of nodes, and Wᵢⱼ has the value 1 if there is a relationship between nodes i and j and 0 otherwise, and D as a matrix with the main diagonal

$${D}_{ii} = {\sum }_{j = 1}^{n} {W}_{ij}$$

and 0 for all other entries. Following this, the similarity measure Simₙₒᵣₘ(x,y) between two regions or images x and y is defined as the element S_{xy} of the matrix S, and is represented as

$${Sim}_{norm} ( x , y ) = {S}_{xy} = \frac{{W}_{xy}}{\sqrt{{D}_{xx} {D}_{yy}}} \qquad ( 5 )$$

For cervical tissue type, various multivariate similarity measures for diagnostic features can be applied.

Content-based Similarity: For content-based similarity, the following measures are preferably employed.

Contents similarity (Qi, X., Nie, L., and Davison, B. D., “Measuring similarity to detect qualified links,” Proceedings of 3′^{d} International Workshop on Adversarial Information Retrieval for the Web (AIRWeb), pp. 45-56, 2007: incorporated herein by reference) reflects, as the name implies, the contents similarity of two images. Suppose that there are n features t₁ through tₙ. Then, each image x can be represented by a probability distribution vector vₓ=└v_{x,1},v_{x,2}, . . . , v_{x,n}┘, where each component v_{x,i} is the probability of that region or image xis represented by feature tᵢ. The contents similarity Sim_{topic}(x,y) of two regions or images x and y can then be determined according to:

$${Sim}_{topic} ( x , y ) = {\sum }_{i = 1}^{n} {v}_{x , i} \times {v}_{y , i} \qquad ( 6 )$$

Cosine Similarity Measure (Crandall, D., Cosley, D., Huttenlocher, D., Kleinberg, J., and Suri, S., “Feedback effects between similarity and social influences in online communities,” Proceedings of the 14^{th} International Conference on Knowledge Discover and Data Mining Table of Content, 2008; incorporated herein by reference) is a non-Euclidean distance measure between two vectors, and is commonly used to compare two features in images. Given feature vectors cₓ and c_{y} for two regions or images x and y, the cosine similarity Sim_{cosine}(x,y) of the two regions or images is determined as cosine of the angle θ between the two vectors according to:

$${Sim}_{cosine} ( x , y ) = cos ( θ ) = \frac{{c}_{x} \cdot {c}_{y}}{‖ {c}_{x} ‖ \times ‖ {c}_{y} ‖} \qquad ( 7 )$$

Earth Mover's Distance (EMD) (Levina, E. and Bickel, P., “The EarthMover's Distance is the mallows distance: some insights from statistics,” Proceedings of International Conference in Computer Vision 2001, pp. 251-256, 2001; incorporated herein by reference) is a distance metric for distributions. It measures the amount of work necessary to fit one distribution to another by moving distribution mass. EMD was originally designed to measure the difference between color histograms with applications in image databases. However, it can be extended to handle more complicated image signatures. Given two histograms H and H′, the L, norm measures the distance between them as follows:

$$d ( H , {H}^{′} ) = {\sum }_{i} \vert {h}_{i} - {h}_{i}^{′} \vert \qquad ( 8 )$$

Here, there are i bins in the histograms and hᵢ is the value of bin i in histogram H. This measure tends to overestimate distances in cases where there is no exact match between bins, as it does not consider the information in neighboring bins. In this case, the weighted L₂ norm is another option:

d²(H,H′)=(\vec{h}−\vec{h}′)′A(\vec{h}−\vec{h}′) (9)

where A is a weighting matrix with an entry for every possible pair of bins. This measure underestimates the distance between distributions which do not have a strong mean. EMD is a solution to the problems with these distance measures.

The intuition behind EMD is to think of one of the distributions as a pile of earth and the other as a set of holes. EMD measures the amount of work needed to fill the holes with earth, assuming there is enough earth available to fill the holes. This EMD problem can be solved using so called linear programming. Given a set S of suppliers or sources (earth) and a set C of consumers or sinks (holes), linear programming minimizes the cost:

$${\sum }_{i ∈ S} {\sum }_{j ∈ C} {c}_{ij} {f}_{ij} \qquad ( 10 )$$

where cᵢⱼ is the cost of sending one unit of flow along the edge from supplier i to consumer j, and fᵢⱼ is the flow sent along that edge. The cost is minimized subject to the following linear constraints:

$${f}_{ij} \geq 0 \\ {\sum }_{i ∈ S} {f}_{ij} = {y}_{j} \\ {\sum }_{j ∈ C} {f}_{ij} \leq {x}_{i} \qquad ( 11 )$$

where yⱼ is the total demand from sink j and xᵢ is the total supply from source i. Thus, the flow along any edge must be non-negative, the flow received at each sink must equal its demand and the output from any source must be less than or equal to its capacity. This definition extends naturally to signatures by defining one signature as the supplier and the other as the producer. After solving the transportation problem, the EMD becomes:

$$EMD ( x , y ) = \frac{{\sum }_{i ∈ S} {\sum }_{j ∈ C} {c}_{ij} {f}_{ij}}{{\sum }_{j ∈ C} {y}_{j}} \qquad ( 12 )$$

The similarity, Sim_{EMD}(x,y), is then defined as the reciprocal of the EMD according to

$${Sim}_{EMD} ( x , y ) = \frac{1}{EMD ( x , y )} = \frac{{\sum }_{j ∈ C} {y}_{j}}{{\sum }_{i ∈ S} {\sum }_{j ∈ C} {c}_{ij} {f}_{ij}} \qquad ( 13 )$$

Thus, EMD applies to a set of distributions, of which the general and cervical-specific image signatures of the present invention can be viewed as those distributions. EMD can also handle signatures of different sizes, which is likely to arise in the present application when one image contains more regions than the other. EMD also avoids quantization problems that arise when using histograms. Furthermore, EMD admits partial matches, which is particularly useful in the present invention as there may be occlusions in some images and thereby blocking parts of the image content. These factors combine to make EMD the most preferred embodiment for the similarity measure in the present invention. The only pitfall with EMD is the performance concern that arises with solving a linear programming problem for each distance computation. However, this can be solved by utilizing highly optimized algorithms (Megiddo, N., “Linear programming in linear time when the dimension is fixed,” Journal of the ACM 31(1), pp. 114-127, 1984; and Cormen, T. H., Leiserson, C. E., Rivest, R. L., and Stein, C., “Introduction to Algorithms,” The MIT Press, 2001; both incorporated herein by reference), and incorporating multithreading in the database design.

Other methods, such as Integrated Region Matching (IEM) (Li, J., Wang, J. Z., and Wiederhold, G., “IRM: integrated region matching for image retrieval,” Proceedings of the 8th ACM International Conference on Multimedia, pp, 147-156, 2000; incorporated herein by reference), that are closely related to ERM but eliminate the need for linear programming can also be used. Furthermore, methods based on relative differential entropy (Cover, T. M., and Thomas, J. A., “Elements of Information Theory,” Wiley Interscience, New York, 1991), and local interest point detectors such as SIFT and SURF (Sargent, D., Chen, C.-I., Tsai, T., Koppel, D., and Wang, Y.-F., “Feature detector and descriptor for medical images,” Proc. SPIE 7259, pp. 72592Z-1—8, 2009, both incorporated herein by reference) can also be employed as similarity measures.

Combination of Similarities: The relation- and content-based similarity measures are combined to capture both neighborhood and content relationships between the local features in the segmented images.

One simple and straightforward method to combine two methods is using a weighted sum. With this method, the final similarity measure Sim_{final}(x,y) between regions or images x and y is a linear combination of a relation-based measure Simᵣₑₗₐₜᵢₒₙ(x,y) and a content-based measure Sim_{content}(x,y) according to

Sim_{final}(x,y)=α·Simᵣₑₗₐₜᵢₒₙ+(1−α)·Sim_{content} (14)

where 0≦α≦1 is a coefficient and provides the mean to weight the importance of the different similarity measures.

Another approach of combining similarity measures is to apply learning algorithms such as Support Vector Machines, but any type of machine learning can be used. With k different similarity measures a vector composed of the different similarity measures can be defined. Through learning, a combined similarity score can be determined, providing a measure of confidence that two images are diagnostically close.

Expansion of Similarity Measures: As with the image signature, the integration of cervical tissue types and diagnostic features, obtained either by image processing and detection algorithms or annotations, into the similarity measure are straightforward. Due to the general-to-specific region-based design which represents an image as a set of vectors, the cervical-specific features merely add more vectors into the image signature, which will not necessitate any changes to the design of the similarity measure. In the preferred embodiment of the present invention, tissue types are preferably compared using a relation-based similarity measure to take into account similarities between neighborhood tissue types. Diagnostic features are preferably compared using the content of the features.

The expansion requires the determination of an optimal weighting to assign to the cervical feature vectors. This weighting should emphasize the importance of the cervical-specific features while not overwhelming the weighting of the general features, so that the general-to-specific system architecture is maintained. To select the weights, the Expectation-Maximization (EM) algorithm (Carson, C., Thomas, M., Belongie, S., Hellerstein, J. M., and Malik, J., “Blobworld: A system for region-based image indexing and retrieval,” Lecture Notes in Computer Science, pp. 509-516, 1999; and Carson, C., Belongie, S., Greenspan, H., and Malik, J., “Blobworld: image segmentation using expectation-maximization and its application to image querying,” IEEE Transactions on Pattern Analysis and Machine Intelligence 24(8), pp. 1026-1038, 2002; both incorporated herein by reference) is preferably applied. Other weighting approaches producing similar results can preferably also be used.

Clustering

With a large set of images in a database, clustering of the images into clusters is required to generalize annotations and image processing results and to label images without annotations and algorithm results. In a database, some images will labeled with keywords, annotations, or image processing results, while the other images will be partly labeled, or not labeled at all. The preferred embodiment of the present invention applies semi supervised learning via normalized graph cut clustering to generalize from the labeled images in order to learn and apply labeling to the remaining unlabeled images. The approach preferably uses a simultaneous k-partition algorithm based on normalized graph cut clustering that is extended to incorporate semi-supervised learning.

Normalized Graph Cut—Given a set of N data points to cluster, the points can be considered as a set of vertices V in a graph G, with edges between the vertices weighted by the similarity between the corresponding data points. An optimal bipartition of the data can be produced by a graph cut that maximizes the intra-cluster similarity while minimizing the inter-cluster similarity. An optimal clustering of this type is given by the minimum cut in G; that is, the minimum weight set of edges that, when removed, partition G into two subsets A and B. Such a clustering can be produced by any maximum flow algorithm (as, for example, described in Wu, Z. and Leahy, R., “An optimal graph theoretic approach to data clustering: theory and its application to image segmentation,” IEEE Transactions of Pattern Analysis and Machine Intelligence 15(11), pp. 1101-1113, 1993; incorporated herein by reference). However, in practice this method often favors degenerate cuts that remove a single node from the graph (as noted in Joachims, T., “Transductive learning via spectral graph partitioning,” International Conference on Machine Learning 20, pp. 290-297, 2003; incorporated herein by reference). A more robust method called normalized cut (Shi, J. and Malik, J., “Normalized cuts and image segmentation,” IEEE Transactions on Pattern Analysis and Machine Intelligence 22(8), pp. 888-905, 2000; incorporated herein by reference) is needed to produce useful partitions.

Normalized cuts avoid unbalanced partitions by using the following cut definition:

Ncut(A,B)=2cut(A,B)/(assoc(A,V)+assoc(B,V)) (15)

where V is the vertex set, 2cut is the weight of the edges crossing the cut from A to B, and assoc(A, V) is the total connection weight from A to the entire vertex set. With this measure, only cuts in which both A and B contain a significant percentage of the vertices will have a low value. Cuts involving a small number of vertices will not be chosen, as 2cut(A, B) will be a large percentage of assoc(A, V) in such cases.

While this definition solves the problem of degenerate cuts, finding an optimal normalized cut is known to be NP-hard (nondeterministic polynomial-time). However, relaxing the problem to allow real numbered solutions instead of hard assignments leads to an objective function of the following form:

(D−W)y=λDy (16)

Here, W is the edge weighting adjacency matrix of the graph, y is the real-valued solution vector, and D=diag(W1_{N}), where 1_{N} is a length N vector of ones. This problem is tractable and can be solved by eigenvalue decomposition. The real-valued assignment vector can then be used either to rank the data points, or a threshold can be set to produce a hard bipartition of the training examples. Although this method only approximates the optimal normalized cut, it produces good results in practice.

Simultaneous k-partition—The normalized graph cut clustering method is an unsupervised clustering method that partitions the input into two clusters. Typically, a k-partition is created by recursively applying the bipartition algorithm. The present invention will instead base a semi supervised learner on a generalization of the above method for simultaneous k-partitioning (Byrne, J., Gandhe, A., Prasanth, R. K., Ravichandan, B., Huff, m., Mehra, R. K., “A k-partition, graph theoretic approach to perceptual organization,” International Conference on Integration of Knowledge Intensive Multi-Agent Systems, p 336-342, 2003: incorporated herein by reference). This method provides better performance and the means to avoid sub-optimal cuts.

The need to specify the number of clusters in advance is a disadvantage of the k-partitioning method. With a large database of images and videos, it may be difficult to estimate the number of clusters needed. For these cases, a generalized Spectral Graph Transducer (SGT) (Joachims, T., Transductive learning via spectral graph partitioning, International Conference on Machine Learning 20, pp. 290-297, 2003; incorporated herein by reference) approach can be applied to the semi supervised learning to handle k-way partitioning through recursive bipartition. This allows for the adaptive selecting of the number of clusters by recursively partitioning the input based on a measure of intra-cluster cohesiveness, such as entropy.

Semi-supervised Learner—The present invention extends normalized graph cut clustering with simultaneous k-partitions by incorporating semi-supervised learning (Joachims, T., “Transductive learning via spectral graph partitioning,” International Conference on Machine Learning 20, pp. 290-297, 2003; incorporated herein by reference). In the present invention weights are used to control the penalty for incorrect labeling and ensure that the final clustering has a low training error. This formulation provides an initial clustering that can be updated incrementally through user feedback (as discussed in a following section).

Further to the method described above, supervised learning methods such as generalized conditional random fields (as described in co-pending, commonly assigned patent application entitled “Cervical cancer detection using conditional random fields,” U.S. patent application Ser. No. 13/068,188 and International patent application #PCT/US2011/000778, both filed May 3, 2011, and both incorporated herein by reference), and hidden Markov models (as described in co-pending, commonly assigned patent application entitled “Versatile video interpretation, visualization, and management system,” U.S. patent application Ser. No. 13/134,507 and International patent application #PCT/US2011/01051, both filed Jun. 7, 2011, and both incorporated herein by reference), are other clustering methods that can be used.

Classification

With the database images clustered into clusters, the query image can be compared with a representative from each cluster. Then, the top ranked images from the most similar cluster can be returned to the user, along with images from the second best cluster for user feedback. One option for representing a cluster is to average the feature descriptors of all images in the cluster to produce the mean example from that cluster. The query image can then be compared against each cluster mean using the similarity measure discussed previously. This method uses a distance metric, and is equivalent to a linear nearest-neighbor classification. Since real data is unlikely to be linearly separable, a classifier that can describe a more complex decision boundary is preferably used. This can be accomplished by using a kernel method with support vector machine (SVM) classification (Cristianini, N. and Shawe-Taylor, J., “An Introduction to Support Vector Machines and other Kernel-Based Learning Methods,” Cambridge University Press, 2001; and Shawe-Taylor, J. and Cristianini, N., “Kernel Methods for Pattern Analysis,” Cambridge University Press, 2004; both incorporated herein by reference) using all the features in the feature extraction portion of the image signature process described above.

Support Vector Machine—The SVM is a supervised learning method that learns a linear classification boundary between a set of positive and negative training examples. The SVM decision boundary is determined as the solution to the following quadratic programming problem:

min_{w,b}½∥w∥²,cᵢ(w·xᵢ−b)≧1 (17)

where xᵢ is the i^{th} training example, cᵢ is its class(1 or −1), w is the normal to the decision boundary, b is the offset of the decision boundary from the origin, and ‘·’ represents the dot product operation. The solution to this problem is an optimal hyperplane, in the sense that the margin of separation between positive and negative examples is maximized. The decision boundary is determined by the support vectors, or training examples closest to the decision boundary. Once training is complete, a test example can be classified using a single dot product, checking which side of the boundary it lies on.

The above discussion describes a hard margin SVM, which is only applicable if the data is linearly separable. In reality, due to noise and other factors, it is unlikely that cervical image features will be linearly separable. In this case, a soft-margin SVM can be used, and which allows some training examples to be misclassified according to a penalty term added to the quadratic programming objective function as follows:

$${min}_{w , b} \frac{1}{2} {‖ w ‖}^{2} + C {\sum }_{i = 1}^{N} {ξ}_{i} , {c}_{i} ( w \cdot {x}_{i} - b ) \geq 1 - {ξ}_{i} \qquad ( 18 )$$

The new variables ξᵢ are called slack variables and are included to allow examples to be misclassified, while C is a constant controlling the misclassification penalty. This formulation is used to account for noisy input data and mislabeled training examples from clustering. This soft-margin SVM is then extended to provide nonlinear decision boundaries via kernel methods.

Multi-class SVM with Kernel Methods—By applying SVMs with different kernels, a nonlinear classification boundary can be achieved by mapping the training examples into a higher dimensional space. The goal of such an operation is to map data which are not linearly separable into a space in which they become linearly separable. To handle the classification of features, clusters and images into multiple classes, a one-versus-all soft-margin SVM is trained to represent each class c, treating examples from class c as positive and all remaining examples as negative. A query is answered using maximum likelihood, classifying the query image using each one-versus-all SVM and designating the SVM with the highest output as the winner.

Further to the method described above, an approach in which features, clusters or images are represented as eigenimages (Abadpour, A. and Kasaei, S., “Color PCA eigenimages and their application of compression and information retrieval,” Image and Vision Computing 26(7), pp. 878-890, 2008; incorporated herein by reference) could also be used to compare query images to eigenimage representatives from each cluster. The most similar eigenimage will then correspond to the winning cluster

User Relevance Feedback and User-Specified Search Metrics

User Relevance Feedback: In content-based image retrieval, complex interactions between users, the system, and semantic interpretations guide the retrieval approach. Image retrieval based on users' responses is the repeatable process and by capturing users' search intentions and modifying search strategies the accuracy of image retrieval can be improved. Two different user relevance feedback mechanisms are incorporated into the present invention: keyword search feedback and image search feedback.

Keyword Search Feedback: As keyword searches (query-by-keyword) require image understanding to translate words into visual concepts, and must deal with the many different ways in which a given image can be interpreted, user relevance feedback for keyword searches are preferably considered for expert users only. When an expert user provides keywords for image search, in addition to the normal image search results, the present invention provides separate search results with images with low keyword credibility for relevance feedback. Then, the expert user reviews the resulting images with low keyword credibility, and confirms or rejects the proposed keywords for each image.

A potential problem with user relevance feedback from experts is when they do not agree with each other. This could introduce inconsistencies in the clustering and classification algorithms that could be difficult to resolve. As a solution to this problem, the majority response from experts is considered, and the expert responses are weighted based on, for example, years of experience and disease specialization.

Image Search Feedback: The aim of user relevance feedback for image search (query-by-example-image) is to update the search space representations of the images so that the updated representations will enhance the search results based on users' responses. This can be accomplished according to the following process. Consider the following definitions:

C={C₁,C₂, . . . , C_{K}} (19)

f^{I}=[f₁^{I},f₂^{I}, . . . , fₙ^{I}] (20)

s^{I}=[s₁^{I},s₂^{I}, . . . , sₙ^{I}] (21)

where C is a set of K image classes and f^{I} is an n-dimensional feature vector representing image I. The search space is S, and its dimension |S| is n, the same as the feature space. Each image I that resides in the database is represented by a search vector s^{I}. Initially, before any user feedback, s^{I}=f^{I} is for all images. Let c^{k}(s^{J}) denote the membership function for class k, returning the probability that image I is a member of class k, and δ(s^{I},s^{J}) denote the similarity function between two images I and J. Now, suppose that a user supplies a query image Q. Then, the class C_{Q} for the matching images is determined by:

$${C}_{Q} = {arg max}_{{C}_{k}} {c}^{k} ( {s}^{Q} ) \qquad ( 22 )$$

where s^{Q}=f^{Q}. Let M denote the number of images to be retrieved; then the set of retrieval images, R(Q), is defined as:

R(Q)={I₁^{R},I₂^{R}, . . . , I_{M}^{R}} (23)

R(Q) is determined by selecting the M nearest images to the example image Q in the class C_{Q}, using a similarity function as described previously. Then, the system returns these M images for user feedback. Among the returned images, the user determines which images are and are not relevant to the query. Let P and N denote the set of images that the user selected as relevant and as irrelevant, respectively. Based on the user feedback, the search vectors of the images in the sets P and N are updated using a gradient method as follows:

$${s}_{new}^{I} = \{ \begin{matrix}{s}^{I} + α ∇ δ ( • , {s}^{{I}_{e}} ) , & if I ∈ P , \\ {s}^{I} - β ∇ δ ( • , {s}^{{I}_{e}} ) , & if I ∈ N ,\end{matrix} \qquad ( 24 )$$

where α and β>0.

User-Specified Search Metrics: To provide an extensible solution, the present invention also enables users to extend the system for their specific applications. Two main extensions are preferably provided; search metric and features. First, users will be allowed to define or choose the search metric. In its basic configuration, the system provides pre-defined similarity measures (as previously described) from which the users may select. Furthermore, users can define their own similarity metrics and plug the metrics into the system. This functionality enables the proposed system to be utilized as a diagnostic support system for any specific cancer type in the clinic. Second, users are able to modify or extend the image signatures as well as the tissue types and diagnostic features for search (as previously described). Secondly, users will be able to define and incorporate their own features into the system.

The inclusion of user relevance feedback and user-specified search metrics will extend the system to learn from the users and to satisfy their specific needs. It produces an online learning system that evolves constantly in response to user feedback, keeping the search results current and ensuring that the system continues to produce relevant results in the future. It also adds an advanced search option, allowing the users to refine the search metrics to their needs.

### INDUSTRIAL APPLICATIONS

This invention can be used whenever it is desired to provide a system for diagnostic support to practitioners in the field.

What is claimed is:

1. A diagnosis support system for a user, comprising:
a database containing high-resolution standardized database images that have been clustered into clusters, each cluster having a cluster feature vector computed by image features in regions in said database images in said cluster, using an overlapping clustering algorithm that allows database images to be assigned to more than one cluster; and
a query-by-example image retrieval application that applies a similarity measure between a query feature vector computed by image features in regions in a query image, and said cluster feature vectors;
wherein said feature vectors are automatically computed by quantitatively describing image signatures for said images by:
image segmentation into regions; and
feature extraction of image features in said regions to compute said feature vectors;
wherein said query-by-example image retrieval application returns a list of database images similar to said query image, ranked by similarity.

2. A diagnosis support system according to claim 1, wherein said database images have been clustered by cluster feature vectors that have been computed using features that are most discriminative between cluster feature vectors.

3. A diagnosis support system according to claim 1, wherein said similarity measure comprises a combination of similarity measures selected from the group consisting of linear combination, linear nearest neighbor classification, and support vector machine.

4. A diagnosis support system according to claim 1, wherein said query-by-example image retrieval application classifies said query image with labels from the cluster of the most similar representative database image as determined by said similarity measure.

5. A diagnosis support system according to claim 1, wherein said image features further comprise tissue types and diagnostic features selected from the group consisting of anatomical features, vessels, acetowhite color and opacity, lesion margins, CIN 1, CIN 2, CIN 3, CIS, and invasive carcinoma.

6. A diagnosis support system according to claim 1, wherein said clustering was performed by using a process selected from the group consisting of semi supervised learning via normalized graph cut clustering, generalized conditional random fields and hidden Markov models, to provide clusters of said database images.

7. A diagnosis support system according to claim 1, wherein meta-data is associated with at least some of said database images, wherein said meta-data includes keywords and annotations.

8. A diagnosis support system according to claim 1, wherein said cluster feature vector is computed by image features from the mean example in the cluster.

9. A diagnosis support system according to claim 1, wherein said query-by-example image retrieval application returns representative database images from the most similar cluster to said user, together with representative database images from the second best cluster for user feedback.

10. A diagnosis support system according to claim 9, wherein said user feedback includes keyword feedback relating to keywords associated with said returned representative database images and image search feedback relating to similarity of said returned representative database images.

11. A diagnosis support system according to claim 10, wherein said keyword feedback is provided by expert users who confirm or reject proposed keywords for said representative database images.

12. A diagnosis support system according to claim 10, wherein said image search feedback comprises updating search vectors of said returned representative database images based on said user's evaluation of the relevance of said returned representative database images.

13. A diagnosis support system according to claim 1, wherein said similarity measure comprises:
a relation-based similarity measure selected from the group consisting of Dice's coefficient, Jaccard's similarity coefficient, normalized adjacency matrix, and multivariate similarity measures; and
a content-based similarity measure selected from the group consisting of Jaccard's similarity coefficient, Contents similarity, Cosine Similarity Measure, Earth Mover's Distance, Integrated Region Matching, relative differential entropy, and local interest point detectors;
wherein said relation-based similarity measure and said content-based similarity measure are combined using a method selected from the group consisting of weighted sum and learning algorithms.

14. A diagnosis support system according to claim 1, wherein:
said local features of said regions comprise color, texture and shape.

15. A diagnosis support system according to claim 1, wherein said database also contains user-defined imagery and user-defined annotations.

16. A diagnosis support system according to claim 7, further comprising:
a text search application to query said database based on text in said meta-data.

17. A diagnosis support system according to claim 1, further comprising:
a query-by-keyword image retrieval application that retrieves selected database images based on keywords associated with said selected database images.

18. A diagnosis support system according to claim 1, wherein said clustering algorithm does not specify the number of clusters in advance.

19. A diagnosis support system according to claim 1, further comprising an information center in communication with said database and said user, wherein experts can review said query image and provide a diagnosis.

20. A process for providing diagnosis support to a user, comprising:
providing a database containing high-resolution standardized database images, wherein at least some of said database images are unlabeled;
presenting a query image;
automatically computing feature vectors by image features in regions in said images by quantitatively describing image signatures for said images, by:
segmenting said images into regions; and
extracting features from said regions to produce feature vectors;
clustering said database images into clusters, each cluster having a cluster feature vector computed by image features in regions in said database images in said cluster;
retrieving database images similar to said query image by applying a similarity measure between said feature vector for said query image and said cluster feature vectors; and
returning a list of images similar to said query image, ranked by similarity.

21. A process for providing diagnosis support to a user, according to claim 20, wherein said clustering step is performed using semi supervised learning via normalized graph cut clustering to provide clusters of said database images.

22. A process for providing diagnostic support to a user, according to claim 20, wherein said returning step comprises returning a representative database image from the most similar cluster to said user, together with a representative database image from the second best cluster for user feedback.

23. A process according to claim 20, wherein said database has meta-data associated with at least some of said database images, wherein said meta-data includes keywords and annotations.
